(19) United States
(12) Patent Application Publication
(10) Pub. No.: US 2023/0114939 A1
(43) Pub. Date: Apr. 13, 2023

(54) SULFATE FREE SHAMPOO COMPOSITION CONTAINING A CATIONIC POLYMER AND INORGANIC SALT

(71) Applicant: The Procter & Gamble Company, Cincinnati, OH (US)

(72) Inventor: Brooke Michele Cochran, Cincinnati, OH (US)

(21) Appl. No.: 17/960,865

(22) Filed: Oct. 6, 2022

Related U.S. Application Data
(60) Provisional application No. 63/253,355, filed on Oct. 7, 2021.

(51) Int. Cl. A61K 8/73; A61K 8/19; A61K 8/46; A61K 8/42; A61Q 5/02

(52) U.S. Cl. CPC A61K 8/737; A61K 8/19; A61K 8/731; A61K 8/466; A61K 8/42; A61Q 5/02

(57) ABSTRACT

A stable shampoo composition that is substantially free of sulfated surfactants and lacks an in situ coacervate. The shampoo composition contains an inorganic salt and a cationic polymer having a charge density greater than 0.6 meq/g. The shampoo composition has a ratio of polymer charge density to inorganic salt of 1.2:1 to 2:1. The shampoo composition can be stable and can lack in situ coacervate.

### FIELD

The present invention relates to a shampoo composition, in particular a conditioning shampoo composition with a cationic polymer having a charge density greater than 0.6 meq/gm and an inorganic salt.

### BACKGROUND

Consumers use shampoo to remove dirt and oil from the surface of hair fibers and scalp. In traditional shampoo compositions, this cleaning is generally provided by incorporating a surfactant system that contains sulfate-based anionic surfactants (e.g. sodium lauryl sulfate, sodium laureth sulfate) into the shampoo composition. These traditional shampoo compositions are easy to apply because they have a viscosity such that the shampoo can be dispensed into an open palm and then spread across the user's hair and scalp. Another advantage of shampoos with sulfate-based surfactants is that they can be paired with cationic polymers that form a coacervate when diluted with water during use that is deposited onto the hair to provide a conditioning benefit.

Recently, many consumers, especially those with color-treated or otherwise treated hair, may prefer a shampoo with a sulfate-free surfactant system. These consumers may also want conditioning polymers in their shampoo because higher conditioning shampoos feel less stripping to the hair. However, it can be difficult to formulate a shampoo with a sulfate-free surfactant system that also has cationic polymers that deliver a conditioning benefit because the shampoo can be unstable. Sulfate-free shampoos can have a relatively high level of inorganic salt, since inorganic salts are a common byproduct in the synthesis of these types of surfactants. In addition, inorganic salts are commonly added to shampoo compositions to help increase the viscosity. However, sulfate-free shampoo compositions that contain a cationic polymer(s) and a relatively high salt content can form an undesirable in situ coacervate phase prior to use, rather than during use, which is desired. The in situ coacervate can separate resulting in inconsistent in use performance and the product can appear cloudy and/or with a precipitated layer.

It is possible to decrease the amount of inorganic salt in the sulfate-free shampoo composition to prevent the in situ coacervate from forming. However, this can cause the viscosity of the shampoo composition to decrease too much, making it difficult to hold in a user's palm and apply across the hair and scalp.

Therefore, there is a need for a stable shampoo product with a sufficient viscosity and superior product performance that contains a surfactant system that is substantially free of sulfate-based surfactants, cationic polymers, and inorganic salts without forming the in situ coacervate phase in the product prior to dilution with water.

### SUMMARY

A shampoo composition comprising: (a) 3% to 35% of an anionic surfactant; wherein the anionic surfactant is substantially free of sulfated surfactants; (b) 5% to 15% of an amphoteric surfactant; (c) an inorganic salt; (d) 0.01% to 2% of a cationic polymer having a charge density greater than 0.6 meq/g; wherein the shampoo has a ratio of polymer charge density to inorganic salt of 1.2:1 to 2:1.

A stable shampoo composition comprising: (a) 3% to 35% of an anionic surfactant selected from isethionates, sarcosinates, and combinations thereof; (b) 5% to 15% of an amphoteric surfactant selected from cocamidopropyl betaine, lauramidopropyl betaine, and combinations thereof; wherein the ratio of the anionic to the amphoteric surfactant is 0.5:1 to 1.5:1; (c) 0.01% to 2% of a cationic polymer having a charge density greater than 0.6 meq/g; (d) greater than 0.6% sodium chloride; wherein the composition is substantially free of sulfated surfactants; wherein the shampoo has a ratio of polymer charge density to inorganic salt of 1.2:1 to 2:1; wherein the shampoo composition lacks in situ coacervate, as determined by the Microscopy Method to Determine Lack of In Situ Coacervate.

### BRIEF DESCRIPTION OF THE DRAWINGS

FIG. 1 is a 20× micrograph showing a marketed shampoo composition that contains an in situ coacervate.

FIG. 2 is a 10× micrograph of the shampoo composition shown in FIG. 1.

FIG. 3 is a photograph of Comparative Example 2, nine months after making.

### DETAILED DESCRIPTION

At least some consumers prefer a shampoo composition that uses a sulfate-free surfactant system and contains a cationic conditioning polymer, which tends to make the shampoo feel less stripping to the hair. However, such a shampoo may exhibit instability if it has a relatively high salt content, which is common in conventional conditioning shampoos. In particular, the inorganic salt in a conditioning shampoo composition can form an undesirable in situ coacervate prior to use (referred to herein as “in situ coacervate” or an “in situ coacervate phase”). Rather, the coacervate should be formed during use when the shampoo composition is diluted with water. The in situ coacervate that forms prior to dilution can cause inconsistent product performance, a cloudy appearance in the composition, and/or the formation of a precipitate layer.

The formation of coacervate upon dilution of the cleansing composition with water during use, rather than while in the bottle on the shelf, is important to improving wet conditioning and deposition of various conditioning actives, especially those that have small droplet sizes (i.e., ≤2 microns). One way to form good quality coacervate at the right time (upon dilution during use), is to formulate with very low (e.g., <1%) or no salt by limiting the amount of inorganic salt that is added to the composition and that comes in with the surfactant materials. However, inorganic salt helps to elongate micelles to build viscosity. Therefore, these compositions generally have a viscosity that is too low, which is not consumer preferred because it is difficult to use the product.

In some shampoo formulations, the viscosity can be increased by decreasing the pH. However, many sulfate-free surfactant systems can hydrolyze at low pH resulting in viscosity and performance changes over time and will eventually lead to phase separation.

It was found that a stable shampoo composition with an acceptable viscosity and product performance could be made with a surfactant system comprising a sulfate-free anionic surfactant and an amphoteric surfactant, a cationic polymer, and 0.6% or more inorganic salt. It was found that if the shampoo composition had a ratio of polymer charge density to salt of less than 1.2:1, then in situ coacervate can form and if the ratio was greater than 2:1 then the viscosity could be too low to be consumer acceptable.

It was found that formulas containing this higher level of inorganic salt (e.g., greater than or equal to 0.6%) had a higher viscosity than similar formulas that contained lower levels of inorganic salt or formulas that were substantially free or free of inorganic salt. This results from more elongation of surfactant micelles (see Robbins, Clarence. Chemical and Physical Behavior of Human Hair, Springer, Berlin, Germany, 2012, pp. 335. “To control the viscosity of many shampoos, salt is added to the surfactant system. The interaction between salt and long chain surfactants transforms the small spherical micelles of the surfactants into larger rod-like . . . structures that increase the viscosity of the liquid shampoo.”) These higher viscosity formulas may be consumer preferred because it is easier to apply across a user's hair and scalp without it running through their fingers.

Another benefit of the higher viscosity shampoo composition is that since there is more elongation of surfactant micelles, a broader range of formulas with acceptable viscosity can be designed because other formula ingredients are not required to build viscosity. For example, viscosity modifiers, other than an inorganic salt, may not be needed. The composition may be free of or substantially free of viscosity modifiers, other than inorganic salt (e.g., sodium chloride, potassium chloride, sodium sulfate, ammonium chloride, sodium bromide, and combinations thereof), which can include carbomers, cross-linked acrylates, hydrophobically modified associative polymers and cellulose, as described in US Pub. Nos. 2019/0105246 and 2019/010524, incorporated by reference. This can make the shampoo easier to distribute across a user's hair and scalp.

Because acceptable viscosity can be achieved using inorganic salt greater than or equal to 0.6% total inorganic salt content, formulas can be made at a higher pH, which can make the composition more stable and effective due to less surfactant hydrolysis resulting in more consistent viscosity and performance over time.

It can be difficult to formulate stable compositions that include greater than or equal to 0.6% total inorganic salt because inorganic salt in the shampoo composition can come from raw materials and can be added to the formulations. For example, amphoteric surfactants such as betaines typically come with high levels of inorganic salt such as sodium chloride. Use of such high-salt raw materials at levels that bring in greater than 0.6% inorganic salt when summed with added inorganic salt can result in the formation of an undesirable in situ coacervate.

The ratio of polymer charge density to total inorganic salt can be greater than 1.1:1 and less than <15:1. In some embodiments, the ratio of polymer charge density to total inorganic salt is 0.5:1 to 3:1 (e.g., 0.7:1 to 2.5:1, 0.75:1 to 2.25:1, 1:1 to 2:1, 1.2:1 to 1.5:1, 1.1:1 to 1.4:1, or even 1.2:1 to 1.4:1). The ratio of polymer charge density to inorganic salt is the charge density of the cationic polymer (in meq/gm) relative to the wt. % of the inorganic salt, disregarding the units. If the composition contains more than one cationic polymer, then the ratio is calculated according to the polymer with the lowest charge density.

The shampoo compositions herein can have a pH of 4 to 8 (e.g., 4.5 to 7.5, 5 to 7, 5.5 to 6.5, 5.5 to 6, and 6 to 6.5), as determined by the pH Test Method, described herein.

The shampoo composition can include 0.5% to 5% inorganic salt (e.g., 0.55% to 4%, 0.75% to 3.5%, 0.6% to 3.25%, 0.8% to 3%, 0.8% to 2.5%, 1% to 2%, or even 1% to 1.5%). The wt% of inorganic salt can be determine using conventional methods known to those skilled in the art. For example, if the inorganic salt is a chloride salt, then the wt. % inorganic chloride salt can be determined by the Argentometry Method to Measure wt % Inorganic Chloride Salt Test Method, described hereinbelow.

The shampoo composition can have a viscosity of 3000 cP to 20,000 cP, alternatively 4000 cps to 15,000 cps, alternatively from 4500 cP to 12,000 cP, alternatively 5,000 cP to 11,000 cP, and alternatively 7,000 cP to 10,000 cP, as measured at 26.6° C., as measured by the Cone/Plate Viscosity Measurement Test Method, described herein. In some instances, the inorganic salt may be used as a viscosity modifier, alone or in combination with other viscosity modifiers.

It may be consumer desirable to have a shampoo composition with a minimal level of ingredients. The shampoo composition can be formulated without polymeric thickeners or suspending agents such as carbomer, EGDS or thixcin. The shampoo composition may be comprised of 11 or fewer ingredients, 10 or fewer ingredients, 9 or fewer ingredients, 8 or fewer ingredients, 7 or fewer ingredients, 6 or fewer ingredients. The minimal ingredient formula can include water, anionic surfactant, amphoteric surfactant, cationic polymer, inorganic salt, and perfume. It is understood that perfumes can be formed from one or more materials. In some examples, the composition can be free of or substantially free of fragrance. In another example, the composition can be free of or substantially free of PEG.

The shampoo composition can be used to clean and condition hair. First, the user dispenses the liquid shampoo composition from the bottle into their hand or onto a cleaning implement. Then, they massage the shampoo into their wet hair. While they are massaging the shampoo composition into the hair the shampoo is diluted with water causing a coacervate to form and the shampoo to lather. After massaging into hair, the shampoo composition is rinsed from the hair and at least some of the cationic polymer(s) are deposited on the user's hair to provide the hair conditioning benefit. Shampooing can be repeated, if desired, and/or a conditioner can be applied. The conditioner can be a rinse-off conditioner or a leave-in conditioner.

“About” modifies a particular value by referring to a range of plus or minus 20% or less of the stated value (e.g., plus or minus 15% or less, 10% or less, or even 5% or less).

“Cleansing composition” includes personal cleansing products such as shampoos, conditioners, conditioning shampoos, shower gels, liquid hand cleansers, facial cleansers, and other surfactant-based liquid compositions.

“Clear” or “transparent” can be used interchangeably and mean that the composition has a percent transmittance (% T) of at least 80% at 600 nm (e.g., 80% to 100%).

“Fluid” means flowable composition forms such as liquids and gels.

“Molecular weight” (M.Wt.) refers to the weight average molecular weight unless otherwise stated. Molecular weight is measured using industry standard method, gel permeation chromatography (“GPC”). The molecular weight has units of grams/mol.

“Substantially free” means that a material is present in the composition at less than 0.5 wt % (e.g., less than 0.25%, 0.1%, 0.05%, 0.02%, or even less than 0.01%). “Free of” means that there is no detectable amount of a material present in the composition (i.e., 0 wt %).

“Sulfate-free” and variations thereof means the composition is substantially free of or free of sulfate-containing compounds.

“Sulfated surfactants” or “sulfate-based surfactants” means surfactants that contain a sulfate group.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials. Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Surfactant

The cleansing compositions described herein can include one or more sulfate-free surfactants. Surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. The concentration of the sulfate-free surfactant(s) in the composition should be sufficient to provide the desired cleaning and lather performance. For example, the cleansing composition may have a total surfactant level of 5% to 50% (e.g., 8% to 40%, 10% to 30%, 12% to 25%, 13% to 23%, 14% to 21%, 15% to 20%).

The cleansing composition herein includes a surfactant with anionic moieties that can form a coacervate with a suitable cationic polymer. Thus, the surfactants herein can be anionic, amphoteric, zwitterionic, non-ionic, and combinations thereof. Some non-limiting examples of these surfactants are described in U.S. Publication Nos. 2019/0105246 and 2018/0098923, U.S. Pat. No. 9,271,908, and McCutcheon's Emulsifiers and Detergents, 2019, MC Publishing Co.

Cleansing compositions typically employ sulfate-based surfactant systems (such as, but not limited to, sodium lauryl sulfate) because of their effectiveness in lather production, stability, clarity and cleansing. The cleansing compositions described herein are free of or substantially free of sulfate-based surfactants. In some instances, the surfactants can be added to the composition as a solution, instead of a neat material, and the solution can include inorganic salts that can be added to the formula. For example, the inorganic salt may be added to the cleansing composition as a carryover ingredient in the surfactant, up to 2% by weight of the surfactant. In some instances, the surfactant may include less than 1.5% inorganic salt (e.g., less than 1.25%, 1%, 0.7%, 0.5%, 0.25%, 0.2%, 0.15%, or even 0.1% or less).

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof. The anionic surfactant may be present in the cleansing composition at 3% to 30% (e.g., 4% to 20%, 5% to 15%, 6% to 12%, or even 7% to 10%).

In some examples, the surfactant system may include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof. In some examples, the composition may contain an anionic surfactant selected from the group consisting of sulfosuccinates, isethionates, sulfonates, sulfoacetates, glucose carboxylates, alkyl ether carboxylates, acyl taurates, and combinations thereof.

Some non-limiting examples of sulfosuccinate surfactants are disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof. Some non-limiting examples of isethionates are sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof. Some non-limiting examples of sulfonates can include alpha olefin sulfonates, linear alkylbenzene sulfonates, sodium laurylglucosides hydroxypropylsulfonate and combination thereof. Some non-limiting examples of sulfoacetates can include sodium lauryl sulfoacetate, ammonium lauryl sulfoacetate and combination thereof. Some non-limiting examples of glucose carboxylates can include sodium lauryl glucoside carboxylate, sodium cocoyl glucoside carboxylate and combinations thereof. Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof. Non-limiting example of acyl taurates can include sodium methyl cocoyl taurate, sodium methyl lauroyl taurate, sodium methyl oleoyl taurate, sodium caproyl methyl taurate and combination thereof.

The cleansing composition may include 3% to 40% of an amphoteric surfactant (e.g., 4% to 30%, 5% to 25%, 6% to 18%, 7% to 15%, 8% to 13%, or even 9% to 11%). The ratio of anionic surfactant to amphoteric surfactant can be 0.25:1 to 3:1, 0.3:1 to 2.5:1, 0.4:1 to 2:1, 0.5:1 to 1.5:1, 0.6:1 to 1.25:1, and 0.75:1 to 1:1. In some examples, the ratio of anionic surfactant to amphoteric surfactant is less than 2:1, 1.75:1, 1.5:1, 1.1:1, or even less than 1:1. The e amphoteric surfactant can be selected from betaines, sultaines, hydroxysultanes, amphohydroxypropyl sulfonates, alkyl amphoactates, alkyl amphodiacetates, alkyl amphopropionates and combination thereof.

Some non-limiting examples of betaine amphoteric surfactants include coco dimethyl carboxymethyl betaine, cocoamidopropyl betaine (CAPB), cocobetaine, lauramidopropyl betaine (LAPB), coco-betaine, cetyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof. Some non-limiting examples of alkylamphoacetates amphoteric surfactants include sodium cocoyl amphoacetate, sodium lauroyl amphoacetate and combination thereof. Some particularly suitable examples of amphoteric surfactants are cocamidopropyl betaine (CAPB), lauramidopropyl betaine (LAPB), coco-betaine, cetyl betaine and combinations thereof.

The cleansing composition may include one or more non-ionic surfactants selected from alkyl polyglucosides, alkyl glycosides, acyl glucamides and mixture thereof. Some non-limiting examples of alkyl glucosides include decyl glucoside, cocoyl glucoside, lauroyl glucoside and combination thereof. Some non-limiting examples of acyl glucamide include lauroyl/ myristoyl methyl glucamide, capryloyl/ caproyl methyl glucamide, lauroyl/ myristoyl methyl glucamide, cocoyl methyl glucamide and combinations thereof.

The cleansing composition may include a non-ionic detersive surfactant such as, for example, cocamide, cocamide methyl MEA, cocamide DEA, cocamide MEA, cocamide MIPA, lauramide DEA, lauramide MEA, lauramide MIPA, myristamide DEA, myristamide MEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, and mixtures thereof.

### Cationic Polymer

The cleansing composition herein includes a cationic polymer that can form a coacervate with the anionic moieties of the surfactant(s). Some non-limiting examples of cationic polymers that may be suitable for use herein include cationic guar polymers, cationic non-guar galactomannan polymers, cationic starch polymers, cationic copolymers of acrylamide monomers and cationic monomers, synthetic non-crosslinked cationic polymers, which may or may not form lyotropic liquid crystals upon combination with the detersive surfactant, and cationic cellulose polymers. Some non-limiting examples of these cationic polymers are disclosed in U.S. Publication Nos. 2019/0105247 and 2021/0346265.

Some particularly suitable examples of cationic guar polymers include guar hydroxypropyltrimonium chloride such as the Jaguar° series from Solvay® S.A., Hi-Care™ series from Rhodia®, and N-Hance™ and AquaCat™ from Ashland™. Some particularly suitable examples of galactomannan polymer derivative include galactomannan polymers that have a mannose to galactose ratio of greater than 2:1 on a monomer to monomer basis obtained from the endosperm of seeds of the Leguminosae family (e.g., tara gum (3 parts mannose/1 part galactose), locust bean or carob (4 parts mannose/1 part galactose), and cassia gum (5 parts mannose/1 part galactose)). Some particularly suitable examples of cationic starch particles include those with a degree of substitution of 0.2 to 2.5 using substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. The “degree of substitution” of the cationically modified starch polymers is an average measure of the number of hydroxyl groups on each anhydroglucose unit which is derivatized by substituent groups. Some particularly suitable examples of cationic cellulose polymers include salts of hydroxyethyl cellulose reacted with a suitable ammonium substituted epoxide such as polyquaternium 10, polyquaternium 24, and polyquaternium 67. Some non-limiting examples of cationic copolymers of acrylamide monomers and cationic monomers include polyquaternium 76 and trimethylammoniopropylmethacrylamide chloride-N-acrylamide (AM:MAPTAC). Another particularly suitable cationic polymer includes polydiallyldimethylammonium chloride, which is sometimes referred to as poly-DADMAC or polyquaternium 6.

The cationic polymer described herein can also aid in repairing damaged hair, particularly chemically treated hair by providing a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair.

The cationic polymer may be present in the cleansing composition at 0.05% to 3% (e.g., 0.075% to 2.0%, 0.1% to 1.0%, 0.16% to 0.5%, 0.2% to 0.5%, 0.3% to 0.5%, or even 0.4% to 0.5%). The cationic polymers may have a cationic charge density of 0.6 meq/g or more (e.g., 0.9 meq/g, 1.2 meq/g, or 1.5 meq/g or more), but typically less than 7 meq/g (e.g., 2 meq/g-7 meq/g, 3 meq/g-6 meq/g, or even 4 meq/g-5 meq/g). In some examples the composition can include a cationic polymer with charge density of 1.7 to 2.1 meq/g and 1 to 1.5% total inorganic salt. The charge densities can be measured at the pH of intended use of the cleansing composition. (e.g., at pH 3 to pH 9; or pH 4 to pH 8). The average molecular weight of cationic polymers can be between 10,000 Da and 10 million Da (e.g., 50,000 Da to 5 million Da, 100,000 Da to 3 million Da, 300,000 Da to 3 million Da, or even 100,000 Da and 2.5 million Da). Lower molecular weight cationic polymers tend to have greater translucency in the liquid carrier of a cleansing composition.

In some instances, the composition may include a cationic polymer system of 2 or more cationic polymers. For example, the cleansing composition may include a primary cationic polymer that has a charge density of 2 meq/gm to 7 meq/gm (e.g., 3 meq/gm to 7 meq/gm, 4 meq/gm to 7 meq/gm, or even 4.5 meq/gm to 7 meq/gm) and one or more secondary cationic polymers that each have a charge density of 0.6 meq/gm to 4 meq/gm (e.g., 0.6 meq/gm to 2 meq/gm). In some instances, the secondary polymers may form an isotropic floc coacervate upon dilution. The charge density of cationic polymers other than cationic guar polymers can be determined by measuring % Nitrogen according to USP<461>Method II. The % Nitrogen can then be converted to Cationic Polymer Charge Density using calculations known in the art. For cationic guar polymers, the charge density is calculated by first calculating the degree of substitution, as disclosed in WO 2019/096601, and then calculate cationic charge density from the degree of substitution, as described in WO 2013/011122.

Liquid Carrier

As can be appreciated, cleansing compositions can desirably be in the form of pourable liquid under ambient conditions. Inclusion of an appropriate quantity of a liquid carrier can facilitate the formation of a cleansing composition having an appropriate viscosity and rheology. A cleansing composition can include, by weight of the composition, 20% to 95%, by weight, of a liquid carrier, and 60% to 85%, by weight, of a liquid carrier. The liquid carrier can be an aqueous carrier such as water.

Optional Components

As can be appreciated, cleansing compositions described herein can include a variety of optional components to tailor the properties and characteristics of the composition. As can be appreciated, suitable optional components are well known and can generally include any components which are physically and chemically compatible with the essential components of the cleansing compositions described herein. Optional components should not otherwise unduly impair product stability, aesthetics, or performance. Individual concentrations of optional components can generally range 0.001% to 10%, by weight of a cleansing composition. Optional components can be further limited to components which will not impair the clarity of a translucent cleansing composition.

Suitable optional components which can be included in a cleansing composition can include co-surfactants, deposition aids, conditioning agents (including hydrocarbon oils, fatty esters, silicones), anti-dandruff agents, anti-fungal agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, amino acids, skin active agents, sunscreens, UV absorbers, vitamins, and combinations thereof. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

Conditioning Agents

The cleansing composition nay include a synthetic conditioning agent (e.g., silicone conditioning agent), an organic conditioning material such as oil or wax, or a combination of these. The silicone conditioning agent can be a volatile silicone, non-volatile silicone, or a combination thereof. Examples of suitable silicone conditioning agents, and optional suspending agents for the silicone, are described in U.S. Reissue Pat. No. 34,584, U.S. Pat. Nos. 5,104,646, 5,106,609, and 11,116,703.

The organic conditioning agent may be non-polymeric, oligomeric or polymeric. Some non-limiting examples of organic conditioning agents include hydrocarbon oils, polyolefins, fatty esters, fluorinated conditioning compounds, fatty alcohols, alkyl glucosides and alkyl glucoside derivatives, quaternary ammonium compounds, polyethylene glycols and polypropylene glycols having a molecular weight of up to 2,000,000 including those with CTFA names PEG-200, PEG-400, PEG-600, PEG-1000, PEG-2M, PEG-7M, PEG-14M, PEG-45M and mixtures thereof.

Emulsifiers

A variety of anionic and nonionic emulsifiers can be used in the cleansing composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

Chelating Agents

The cleansing composition can may include 0.01% to 10% of a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996). When related to chelants, the term “salts and derivatives thereof” means the salts and derivatives comprising the same functional structure (e.g., same chemical backbone) as the chelant they are referring to and that have similar or better chelating properties. Some non-limiting examples of chelants that may be suitable for use herein are disclosed in U.S. Pat. Nos. 5,747,440 and 5,284,972. Particularly suitable examples of chelants include polymeric ethylenediaminedisuccinic acid (EDDS) and histidine.

Gel Network

The cleansing composition herein may include a fatty alcohol gel network. Gel networks are formed by combining fatty alcohols and surfactants at a suitable ratio (e.g., 1:1 to 40:1, 2:1 to 20:1, or 3:1 to 10:1). The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. Gel networks can provide a number of benefits to cleansing compositions. For example, a gel network can provide a stabilizing benefit to cosmetic creams and hair conditioners. In addition, gel networks can provide conditioned feel benefits to hair conditioners and shampoos.

Some non-limiting examples of gel networks are disclosed in U.S. Pat. No. 10,912,719. In some examples, a gel network can be prepared by charging a vessel with water. In these examples, the water can then be heated to 74° C. A fatty alcohol (e.g., cetyl alcohol and stearyl alcohol) and a surfactant can be added to the heated water. After mixing, the resulting mixture can passed through a heat exchanger where the mixture is cooled to 35° C., which allows the fatty alcohols and surfactant to crystallize and form a crystalline gel network. Table 1 provides the components and their respective amounts for this example.

TABLE 1

| Premix | % |
|---|---|
| Gel Network Surfactant¹ | 11.00 |
| Stearyl Alcohol | 8% |
| Cetyl Alcohol | 4% |
| Water | QS |

¹For anionic gel networks, suitable gel network surfactants above include surfactants with a net negative charge including sulfonates, carboxylates and phosphates among others and mixtures thereof. For cationic gel networks, suitable gel network surfactants above include surfactants with a net positive charge including quaternary ammonium surfactants and mixtures thereof. For Amphoteric or Zwitterionic gel networks, suitable gel network surfactants above include surfactants with both a positive and negative charge at product usage pH including betaines, amine oxides, sultaines, amino acids among others and mixtures thereof.

### Method of Making a Cleansing Composition

A cleansing composition described herein can be formed similarly to known cleansing compositions. For example, the process of making a cleansing composition can include the step of mixing the surfactant, cationic polymer, and liquid carrier together to form a cleansing composition. Additional information on sulfate-free surfactants and other ingredients that are suitable for shampoo compositions is found at U.S. Pub. Nos. 2019/0105247 and 2019/0105246.

### METHODS

### Argentometry Method to Measure Wt % Inorganic Chloride Salts

The weight % of inorganic chloride salt in the composition can be measured using a potentiometric method where the chloride ions in the composition are titrated with silver nitrate. The silver ions react with the chloride ions from the composition to form an insoluble precipitate, silver chloride. The method used an electrode (Mettler Toledeo DM141) that is designed for potentiometric titrations of anions that precipitate with silver. The largest change in the signal occurs at the equivalence point when the amount of added silver ions is equal to the amount of chloride ions in solution. The concentration of silver nitrate solution used should be calibrated using a chloride solution known to one of skill in the art, such as a sodium chloride solution that contains a standard and known amount of sodium chloride to confirm that the results match the known concentration. This type of titration involving a silver ion is known as argentometry and is commonly used to determine the amount of chloride present in a sample.

### Methods to Determine Lack of In Situ Coacervate in Composition Prior to Dilution

### 1. Microscopy Method to Determine Lack of In Situ Coacervate

Techniques for analysis of formation of complex coacervates are known in the art. For example, microscopic analyses of the compositions, at any chosen stage of dilution, can be utilized to identify whether a coacervate phase has formed. Such coacervate phase can be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164.

This method uses a microscope to determine the lack of in situ coacervate. The composition is mixed to homogenize, if needed. Then, the composition is sampled onto a microscope slide and mounted on a microscope, per typical microscopy practices. The sample is viewed at, for example, a 10× or 20× objective. If in situ coacervate is present in the sample, an amorphous, gel-like phase with 20 nm to 200 nm particle size can be seen throughout the sample. This amorphous, gel-like phases can be described as gel chunks or globs. In this method, the in situ coacervate is separate from other ingredients that were intentionally added to the formula that form flocks or otherwise appear as particles under microscopy.

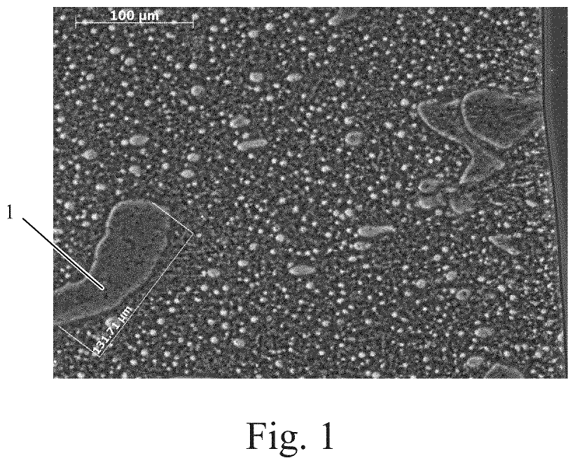

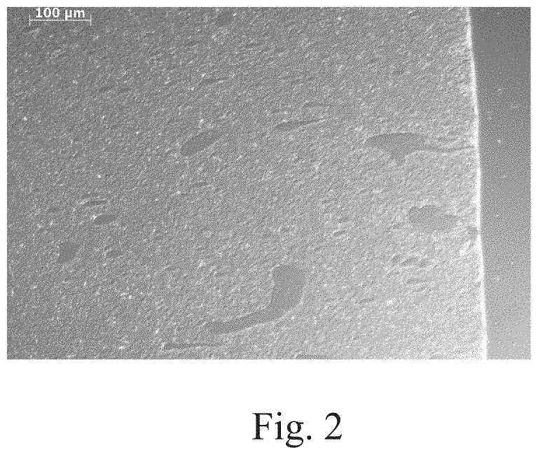

FIG. 1 is an example microscopy photograph at 20× objective of a marketed sulfate-free shampoo composition that contains a cationic polymer and also has in situ coacervate. FIG. 1 at reference numeral 1 shows an amorphous, gel-like phase that is 130 nm long that is the in situ coacervate. FIG. 2 is an example microscopy photograph at 10× objective the same marketed shampoo composition that was used in FIG. 1 at 20× objective. FIG. 2 shows many of these amorphous, gel-like phases present with a length 20 nm to 200 nm.

### 2. Clarity Assessment—Measurement of % Transmittance (% T)

Lack of in situ coacervate can be determined by composition clarity. A composition that does not contain in situ coacervate will be clear, if it does not contain any ingredients that would otherwise give it a hazy appearance.

Composition clarity can be measured by % Transmittance. For this assessment to determine if the composition lacks coacervate, the composition should be made without ingredients that would give the composition a hazy appearance such as silicones, opacifiers, non-silicone oils, micas, and gums or anionic rheology modifiers. It is believed that adding these ingredients would not cause in situ coacervate to form prior to use, however these ingredients will obscure measurement of clarity by % Transmittance.

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple samples are transferred into a 96 well visible flat bottom plate (Greiner part #655-001), ensuring that no air bubbles are within the samples. The flat bottom plate is placed within the SpectraMax M-5 and % T measured using the Software Pro v.5™ software available from Molecular Devices.

### 3. Lasentec FBRM Method

Lack of in situ coacervate can also be measured using Lasentec FBRM Method with no dilution. A Lasentec Focused Beam Reflectance Method (FBRM) [model S400A available from Mettler Toledo Corp] may be used to determine floc size and amount as measured by chord length and particle counts/sec (counts per sec). A composition that is free of flocs can lack in situ coacervate. A composition can have flocs and also be free of in situ coacervate if the flocs are known to be the added particles.

### 4. In Situ Coacervate Centrifuge Method

Lack of in situ coacervate can also be measured by centrifuging a composition and measuring in situ coacervate gravimetrically. For this method, the composition should be made without a suspending agent to allow for separation of an in situ coacervate phase. The composition is centrifuged for 20 minutes at 9200 rpm using a Beckman Couller TJ25 centrifuge. Several time/rpm combinations can be used. The supernatant is then removed and the remaining settled in situ coacervate assessed gravimetrically. % In Situ Coacervate is calculated as the weight of settled in situ coacervate as a percentage of the weight of composition added to the centrifuge tube using the equation below. This quantifies the percentage of the composition that participates in the in situ coacervate phase.

$$\% In Situ Coacervate = \frac{Weight of settled in situ coacervate}{Weight of composition added to centrifuge tube} \times 100$$

### Measures of Improved Performance Due to no In Situ Coacervate Prior to Dilution

The composition does not contain in situ coacervate prior to dilution. Because of this, coacervate quantity and quality upon dilution is better than a composition that does contain in situ coacervate prior to dilution. This provides better wet conditioning and deposition of actives from a composition that does not contain coacervate prior to dilution compared to a composition that does contain coacervate prior to dilution.

### 1. Measurement of % Transmittance (% T) During Dilution

Coacervate formation upon dilution for a transparent or translucent composition can be assessed using a spectrophotometer to measure the percentage of light transmitted through the diluted sample (% T). As percent light transmittance (% T) values measured of the dilution decrease, typically higher levels of coacervate are formed. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1. By averaging the % T values for samples that span a range of dilution ratios, it is possible to simulate and ascertain how much coacervate a composition on average would form as a consumer applies the composition to wet hair, lathers, and then rinses it out. Average % T can be calculated by taking the numerical average of individual % T measurements for the following dilution ratios: 2:1, 3:1, 5:1, 7.5:1, 11:1, 16:1, 24:1, and 34:1. Lower average % T indicates more coacervate is formed on average as a consumer applies the composition to wet hair, lathers and then rinses it out.

% T can be measured using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to insure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple dilution samples can be prepared within a 96 well plate (VWR catalog# 82006-448) and then transferred to a 96 well visible flat bottom plate (Greiner part #655-001), ensuring that no air bubbles are within the sample. The flat bottom plate is placed within the SpectraMax M-5 and % T measured using the Software Pro v.5™ software available from Molecular Devices.

2. Assessment of Coacervate Floc Size upon Dilution

Coacervate floc size upon dilution can be assessed visually. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1. Larger coacervate flocs can indicate a better quality coacervate that provides better wet conditioning and deposition of actives.

### 3. Wet Combing Force Method

Hair switches of 4 grams general population hair at 8 inches length are used for the measurement. Each hair switch is treated with 4 cycles (1 lather/rinse steps per cycle, 0.1 gm cleansing composition/gm hair on each lather/rinse step, drying between each cycle) with the cleansing composition. Four switches are treated with each shampoo. The hair is not dried after the last treatment cycle. While the hair is wet, the hair is pulled through the fine tooth half of two Beautician 3000 combs. Force to pull the hair switch through the combs is measured by a friction analyzer (such as Instron or MTS tensile measurement) with a load cell and outputted in gram-force (gf). The pull is repeated for a total of five pulls per switch. Average wet combing force is calculated by averaging the force measurement from the five pulls across the four hair switches treated with each cleansing composition. Data can be shown as average wet combing force through one or both of the two combs.

### 4. Deposition Method

Deposition of actives can be measured in vitro on hair tresses or in vivo on panelist's heads. The composition is dosed on a hair tress or panelist head at a controlled amount and washed according to a conventional washing protocol. For a hair tress, the tress can be sampled and tested by an appropriate analytical measure to determine quantity deposited of a given active. To measure deposition on a panelist's scalp, the hair is then parted on an area of the scalp to allow an open-ended glass cylinder to be held on the surface while an aliquot of an extraction solution is added and agitated prior to recovery and analytical determination of a given active. To measure deposition on a panelist's hair, a given amount of hair is sampled and then tested by an appropriate analytical measure to determine quantity deposited of a given active.

### Cone/Plate Viscosity Measurement

The viscosities of the examples are measured by a Cone/Plate Controlled Stress Brookfield Rheometer R/S Plus, by Brookfield Engineering Laboratories, Stoughton, Mass. The cone used (Spindle C-75-1) has a diameter of 75 mm and 1° angle. The liquid viscosity is determined using a steady state flow experiment at constant shear rate of 2 s⁻¹ and at temperature of 26.7° C. The sample size is 2.5 ml to 3 ml and the total measurement reading time is 3 minutes.

### Lather Characterization—Kruss DFA100 Lather Characterization

A cleansing composition dilution of 10 parts by weight water to 1 part by weight cleanser is prepared. The shampoo dilution is dispensed into the Kruss DFA100 which generates the lather and measures lather properties.

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe. Submerse the electrode into the testing sample and press the read button. Wait until the pH icon stops flashing and record the value.

### EXAMPLES

The following Examples illustrate various shampoo compositions. In the tables below, inventive Examples 1, 2, 4, and 6-9 and Comparative Examples 1-4 are prepared by conventional formulation and mixing techniques and Examples 3, 5, and 10-11 could be prepared by conventional formulation and mixing techniques.

The total sodium chloride in the tables below is calculated based on the product specifications from the suppliers. Some of the surfactants used in the examples below are sourced in a liquid mixture containing the surfactant at some active concentration, water, and often sodium chloride at some level generated during synthesis of the surfactant. For example, a common surfactant synthesis that produces sodium chloride as a byproduct is the synthesis of cocamidopropyl betaine. In this synthesis, an amidoamine is reacted with sodium monochloroacetate to produce betaine and sodium chloride. This is one example of a surfactant synthesis that produces sodium chloride as a byproduct. Public supplier documents including example Certificate of Analysis and Technical Specification documents list activity by wt % or solids by wt % and wt % sodium chloride. Using these specifications and the surfactant activity in the composition, inherent levels of sodium chloride coming in with the surfactants can be summed up for a given composition and added to any sodium chloride that is directly added to the composition. While surfactants are a common raw material that introduces sodium chloride into the formula, other materials can also be checked for content of sodium chloride to include in the overall sodium chloride calculation. For calculation of total inorganic salt, this total sodium chloride is added to any other inorganic salts that are added through a raw material or intentionally.

The ratio of anionic surfactant to amphoteric surfactant is calculated by wt. %. The ratio of polymer charge density to inorganic salt is the charge density of the polymer (meq/gm) to the wt. % of the inorganic salt, disregarding the units. If the composition contains more than one cationic polymer, then the ratio is calculated according to the polymer with the lowest charge density.

Shampoo compositions with surfactant systems that are substantially free of sulfate-based surfactants can have low viscosity, which makes it more difficult to apply across a user's hair and scalp without it running through their fingers. The viscosity in Table 2Table 2 and Table 3Table 3 was determined using the Cone/Plate Viscosity Measurement Test Method, described herein.

For Examples 1, 2, 4, and 6-9 and Comparative Examples 1-4, the in situ coacervate was determined as follows. The examples were prepared as described herein. The example was made and immediately put in a clear, glass jar of at least 1 inch width. The cap was screwed on the jar, finger tight. The example was stored at ambient temperatures (20-25° C.), away from direct sunlight, for 5 days. For some examples, the composition was stored for up to 9 months to determine if there was phase separation. Then the composition was inspected to see if either haze or precipitate was visually detectable. If either haze or precipitate were present, it was determined that the composition had in situ coacervate. If neither haze nor precipitate were present, it was determined that there was no in situ coacervate. It is believed that the shampoo product would have improved conditioning performance as compared to examples where in situ coacervate formed.

The example was inspected to determine if haze could be visually detected. If the example was clear, then there was no in situ coacervate and it is believed that the shampoo product would have improved conditioning performance as compared to examples where in situ coacervate formed. If haze was detected in the example, then there was in situ coacervate and it is believed that the example would be less preferred by consumers.

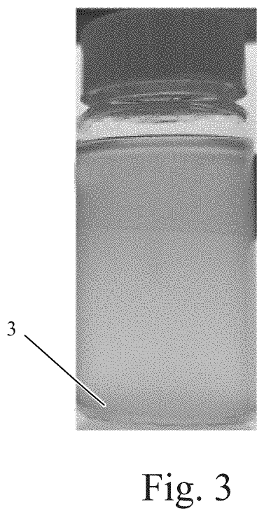

The example was also inspected to determine a separated phase formed on the bottom of the jar. This phase will form in as short as 3 days but could take up to 9 months depending on the viscosity of the composition. FIG. 3 is a photograph of Comparative Example 2 (C2) after 9 months of storage. Reference numeral 3 is a separated coacervate phase at the bottom of the jar.

As used herein, “visually detect” or “visually detectable” means that a human viewer can visually discern the quality of the example with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

The examples in Table 2 to Table 5Table 5, could also be formulated with silicones, opacifiers (e.g. glycol distearate, glycol stearate), non-silicone oils, micas, gums or anionic rheology modifiers and other ingredients that would cause the shampoo to have a hazy appearance. However, it is believed that adding these ingredients would not cause in situ coacervate to form prior to use.

TABLE 2

| | Ex. 1 | C1 | C2 |
|---|---|---|---|
| | (wt. %) | (wt. %) | (wt. %) |
| Lauramidopropyl | 2.44 | 9.75 | — |
| Betaine ¹ | | | |
| Cocamidopropyl Betaine ¹⁰ | 7.31 | — | 9.75 |
| Total Amphoteric Surfactant | 9.75 | 9.75 | 9.75 |
| Sodium Cocoyl | 6.00 | 6.00 | 6.00 |
| Isethionate ² | | | |
| Sodium Lauroyl | 2.5 | 2.5 | 2.5 |
| Sarcosinate ³ | | | |
| Polyquaternium-10 ⁴ (KG- | 0.4 | 0.55 | 0.55 |
| 30M, CD 1.9 meq/g) | | | |
| Sodium Benzoate ⁸ | 0.75 | 0.75 | 0.75 |
| Sodium Salicylate ⁹ | 0.45 | 0.45 | 0.45 |
| Tetrasodium EDTA ¹¹ | 0.16 | 0.16 | 0.16 |
| Silicone ¹² | 0.25 | — | 0.95 |
| Perfume | 1.2 | 1.1 | 1.2 |
| Added Sodium Chloride ⁶ | 0 | 0 | 0.3 |
| Citric Acid | To pH 5.5 to 6.0 | | |
| Water | Q.S. to 100 | | |
| Ratio of Anionic to | 0.9:1 | 0.9:1 | 0.9:1 |
| Amphoteric Surfactant | | | |
| Total Sodium Chloride | 1.3 | 0.07 | 2.0 |
| (including from surfactant) | | | |
| Ratio of Polymer Charge | 1.5:1 | 27:1 | 0.9:1 |
| Density to Inorganic Salt | | | |
| Viscosity | 6561 cP | 2633 cP | 9212 cP |
| Micelle Elongation | Sufficient | Insufficient | Sufficient |
| Contains in situ coacervate | No | No | Yes |
| prior to Dilution? | | | |

Example 1 had 1.3% total sodium chloride and a ratio of polymer charge density to inorganic salt of 1.5:1. No in situ coacervate prior to dilution was observed. The viscosity of Example 1 was measured, and it was determined to be sufficient, and is would be acceptable to consumers. Therefore, it was presumed that the micelle elongation in Example 1 was also sufficient. It is presumed that Example 1 would have good product performance and would be consumer preferred.

As compared to Example 1, Comparative Example 1 (C1) had less total sodium chloride (0.07%) and a ratio of polymer charge density to inorganic salt that was much greater (28:1). C1 also had a viscosity that was significantly lower than Example 1 and it is presumed that C1 did not have the sufficient micelle elongation. It is believed that since C1 has a low viscosity, it would not be preferred by most consumers.

Comparative Example 2 (C2) had 2% total sodium chloride and a ratio of polymer charge density to inorganic salt of 0.9:1. Based on the high viscosity, it is presumed that C2 would have sufficient micelle elongation. However, C2 is expected to have inferior conditioning performance because it contains coacervate that formed during storage, prior to dilution, as seen in FIG. 3.

TABLE 3

| | Ex. 2 | C3 | C4 |
|---|---|---|---|
| | (wt. %) | (wt. %) | (wt. %) |
| Lauramidopropyl | 6.34 | 9.75 | — |
| Betaine ¹ | | | |
| Cocamidopropyl Betaine ¹⁰ | 3.41 | — | 9.75 |
| Total Amphoteric Surfactant | 9.75 | 9.75 | 9.75 |
| Sodium Cocoyl | 6.00 | 6.00 | 6.00 |
| Isethionate ² | | | |
| Sodium Lauroyl | 2.5 | 2.5 | 2.5 |
| Sarcosinate ³ | | | |
| Polyquaternium-10 ⁵ (JR- | 0.4 | 0.25 | 0.55 |
| 30M, CD 1.25 meq/g) | | | |
| Sodium Benzoate ⁸ | 0.75 | 0.75 | 0.75 |
| Sodium Salicylate ⁹ | 0.45 | 0.45 | 0.45 |
| Tetrasodium EDTA ¹¹ | 0.16 | 0.16 | 0.16 |
| Silicone ¹² | 0.25 | 0.25 | 0.25 |
| Perfume | 1.2 | 1.1 | 1.2 |
| Added Sodium Chloride ⁶ | — | — | — |
| Citric Acid ¹⁶ | To pH 5.5 to 5.9 | | |
| Water and Optional | Q.S. to 100 | | |
| Components | | | |
| Ratio of Anionic to | 0.9:1 | 0.9:1 | 0.9:1 |
| Amphoteric Surfactant | | | |
| Total Sodium Chloride | 0.64 | 0.07 | 1.7 |
| (including from surfactant) | | | |
| Ratio of Polymer Charge | 2.0:1 | 18:1 | 0.73:1 |
| Density to Inorganic Salt | | | |
| Viscosity | 3348 cP | 893 cP | 10,116 cP |
| Micelle Elongation | Sufficient | Insufficient | Sufficient |
| Contains in situ coacervate | No | No | Yes |
| prior to Dilution? | | | |

Example 2 had 0.64% total sodium chloride and a ratio of polymer charge density to inorganic salt of 2.0:1. No in situ coacervate prior to dilution was observed. The viscosity of Example 2 was measured, and it was determined to be sufficient, and is would be acceptable to consumers. Therefore, it was presumed that the micelle elongation in Example 2 was also sufficient. It is presumed that Example 2 would have good product performance and be consumer preferred.

As compared to Example 2, Comparative Example 3 (C3) had less total sodium chloride (0.07%) and a ratio of polymer charge density to inorganic salt that was much greater (18:1). C3 also had a viscosity that was significantly lower than Example 2 and it is presumed that C3 did not have the sufficient micelle elongation. It is believed that since C3 has a low viscosity, it would not be preferred by most consumers.

Comparative Example 4 (C4) had 1.7% total sodium chloride and a ratio of polymer charge density to inorganic salt of 0.73:1. Based on the high viscosity, it is presumed that C4 would have sufficient micelle elongation. However, C4 is expected to have inferior conditioning performance because it contains coacervate that formed during storage.

TABLE 4

| | Ex. 3 | Ex.4 | Ex. 5 | Ex. 6 | Ex. 7 | Ex. 8 | Ex. 9 |
|---|---|---|---|---|---|---|---|
| | (wt. %) | (wt. %) | (wt. %) | (wt. %) | (wt. %) | (wt. %) | (wt. %) |
| Lauramidopropyl Betaine ¹ | 2.44 | 2.44 | — | 2.44 | 2.44 | 2.44 | — |
| Low Salt Cocamidopropyl | — | — | 9.75 | — | — | — | — |
| Betaine ⁷ | | | | | | | |
| Cocamidopropyl Betaine ¹⁰ | 7.31 | 7.31 | — | 7.31 | 7.31 | 7.31 | 7.5 |
| Sodium Cocoyl | 6.00 | 6.00 | 6.00 | 6.00 | 6.00 | 6.00 | 4.5 |
| Isethionate ² | | | | | | | |
| Sodium Lauroyl | 4 | — | 2.5 | 2.5 | 2.5 | 2.5 | — |
| Sarcosinate ³ | | | | | | | |
| Polyquaternium-10 ⁴ | 0.4 | 0.4 | 0.4 | 0.05 | 0.55 | 0.15 | 0.25 |
| (KG-30M, CD 1.9 meq/g) | | | | | | | |
| Piroctone Olamine ¹³ | — | — | — | — | — | 0.5 | — |
| Zinc Pyrithione ¹⁴ | — | — | — | — | — | — | 1 |
| Acrylates Copolymer ¹⁵ | — | — | — | — | — | — | 0.7 |
| Added Sodium Chloride ⁶ | 0 | 0 | 1.2 | 0 | 0.2 | 0 | 0 |
| Citric Acid ¹⁶ | To pH 5.5 to 6.5 | | | | | | |
| Water, Preservatives, Fragrance | Q.S. to 100 | | | | | | |
| and Optional Components | | | | | | | |
| Ratio of Anionic to Amphoteric | 1.0:1 | 0.6:1 | 0.9:1 | 0.9:1 | 0.9:1 | 0.9:1 | 0.6:1 |
| Surfactant | | | | | | | |
| Total Sodium Chloride | 1.3 | 1.3 | 1.3 | 1.3 | 1.5 | 1.3 | 1.3 |
| (including from surfactant) | | | | | | | |
| Ratio of Polymer Charge | 1.5:1 | 1.5:1 | 1.5:1 | 1.5:1 | 1.3:1 | 1.5:1 | 1.5:1 |
| Density to Inorganic Salt | | | | | | | |
| Viscosity (cP) | — | 9292 | — | 4076 | 16,698 | 7573 | 13624 |

Examples 4 and 6-9 were made. The viscosity was determined using the Cone/Plate Viscosity Measurement Test Method, described herein. In addition, there was no in situ coacervate prior to dilution and it is believed that these examples would have good conditioning performance and would be consumer preferred.

Examples 3 and 5 could be made. It is expected that these formulas would have sufficient viscosity and micelle elongation and no in situ coacervate would form prior to dilution. It is believed that Examples 3 and 5 would also be consumer preferred.

TABLE 5

| | Ex. 10 | Ex. 11 |
|---|---|---|
| | (wt. %) | (wt. %) |
| Lauramidopropyl Betaine ¹ | — | 2.44 |
| Cocamidopropyl Betaine ¹⁰ | 7.5 | 7.31 |
| Sodium Cocoyl | 4.5 | 6 |
| Isethionate ² | | |
| Sodium Lauroyl | — | 2.5 |
| Sarcosinate ³ | | |
| Polyquaternium-10 ⁴ | 0.4 | 0.4 |
| (KG-30M, CD 1.9 meq/g) | | |
| Sodium Benzoate ⁸ | 0.75 | 0.45 |
| Sodium Salicylate ⁹ | 0.45 | 0.45 |
| Perfume | 1 | 1 |
| Citric Acid ¹⁶ | to pH 5.5- | to pH 5.5- |
| | 6.5 | 6.5 |
| Water | Q.S. to 100 | Q.S. to 100 |
| Ratio of Anionic to | 0.6:1 | 0.9:1 |
| Amphoteric Surfactant | | |
| Total Sodium Chloride | 1.3 | 1.3 |
| (including from surfactant) | | |
| Ratio of Polymer Charge | 1.5:1 | 1.5:1 |
| Density to Inorganic Salt | | |
| Viscosity | ≥Example 1 | ≥Example 1 |
| Micelle Elongation | Sufficient | Sufficient |
| Contains in situ coacervate | No | No |
| prior to Dilution? | | |

Examples 10 and 11 could be made. It is expected that these formulas would have sufficient viscosity and micelle elongation and no in situ coacervate would form prior to dilution. It is believed that Examples 10 and 11 would also be consumer preferred.

### Suppliers for Examples in Table 2 to Table 5

1. Mackam DAB-ULS available from Solvay. Specification Range: Solids=34-36%, Sodium Chloride=0-0.5%. Average values are used for calculations: Actives=35%, Sodium Chloride=0.25%.
2. Hostapon SCI-85 C available from Clariant
3. SP Crodasinic LS30/NP MBAL available from Croda
4. UCARE Polymer KG-30M available from Dow
5. UCARE Polymer JR-30M available from Dow
6. Sodium Chloride available from Norton International Inc.
7. Dehyton PK 45 from BASF with Sodium Chloride removed, resulting in 33.05% Dry Residue, 0.21% Sodium Chloride
8. Sodium Benzoate available from Kalama Chemical
9. Sodium Salicylate available from JQC (Huayin) Pharmaceutical Co., Ltd
10. Tego Betain CK PH 12 available from Evonik. Specification Range: Actives=28-32%, Sodium Chloride=4.5-6%. Average values are used for calculations: Actives=30%, Sodium Chloride=5.25%.
11. Versene 220 Crystals Chelating Agent available from Dow
12. Xiameter MEM-1872 Emulsion available from Dow (sufficiently low particle size to be clear in shampoo compositions at the levels used)
13. Octopirox available from Clamant
14. Zinc Pyrithione available from Lonza
15. Rheocare TTA available from BASF
16. Citric Acid USP Anhydrous Fine Granular available from Archer Daniels Midland Company

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shampoo composition comprising:
a. about 3% to about 35% of a sulfate-free anionic surfactant;
b. about 5% to about 15% of an amphoteric surfactant;
c. an inorganic salt; and
d. about 0.01% to about 2% of a cationic polymer having a charge density of greater than 0.6 meq/g, wherein the shampoo composition has a ratio of polymer charge density to inorganic salt level of about 1.2:1 to about 2:1.

2. The composition of claim 1, wherein the composition comprises at least about 0.6% inorganic salt.

3. The composition of claim 1, wherein the shampoo composition comprises a % T value of at least about 80.

4. The composition of claim 1, wherein the shampoo composition lacks an in situ coacervate, according to the Microscopy Method to Determine Lack of In Situ Coacervate.

5. The composition of claim 1, wherein the ratio of the anionic to the amphoteric surfactant is about 0.5:1 to about 1.5:1.

6. The composition of claim 1, wherein the composition has a pH of about 5 to about 6.5.

7. The composition of claim 1, wherein the anionic surfactant is selected from sodium, ammonium or potassium salts of isethionates, sodium, ammonium or potassium salts of sulfonates, sodium, ammonium or potassium salts of ether sulfonates, sodium, ammonium or potassium salts of sulfosuccinates, sodium, ammonium or potassium salts of sulfoacetates, sodium, ammonium or potassium salts of glycinates, sodium, ammonium or potassium salts of sarcosinates, sodium, ammonium or potassium salts of glutamates, sodium, ammonium or potassium salts of alaninates, sodium, ammonium or potassium salts of carboxylates, sodium, ammonium or potassium salts of taurates, sodium, ammonium or potassium salts of phosphate esters, and combinations thereof.

8. The composition of claim 1 wherein the cationic polymer has a weight average molecular weight of about 300,000 g/mol to about 3,000,000 g/mol.

9. The composition of claim 8, wherein the cationic polymer is selected from cationic guars, cationic cellulose, cationic synthetic homopolymers, cationic synthetic copolymers, and combinations thereof.

10. The composition of claim 1, wherein the inorganic salt is selected from sodium chloride, potassium chloride, sodium sulfate, ammonium chloride, sodium bromide, and combinations thereof.

11. The composition of claim 1, wherein the amphoteric surfactant is selected from betaines, sultaines, hydroxysultanes, amphohydroxypropyl sulfonates, alkyl amphoactates, alkyl amphodiacetates, and combination thereof.

12. The composition of claim 1, further comprising an antidandruff agent.

13. The composition of claim 12, wherein the antidandruff agent is selected from piroctone olamine, zinc pyrithione, sulfur, selenium sulfide and azoxystrobin, and combinations thereof.

14. The composition of claim 1, wherein the composition has a viscosity of about 3000 cP to about 20,000 cP.

15. The composition of claim 1, wherein the composition is substantially free of silicones.

16. The composition of claim 1, wherein the composition consists of 9 or fewer ingredients.

17. The composition of claim 1, wherein the composition is free of viscosity modifiers other than the inorganic salt.

18. A method for cleaning hair comprising:
a. providing the shampoo composition of claim 1;
b. dispensing the shampoo composition into a palm or a cleaning implement;
c. applying the shampoo composition onto wet hair and massaging the shampoo composition across the hair and scalp; wherein the shampoo composition is diluted forming a coacervate that is deposited onto the hair;
d. rinsing the shampoo composition from the hair.

19. A stable shampoo composition comprising:
a. about 3% to about 35% of an anionic surfactant selected from isethionates, sarcosinates, and combinations thereof;
b. about 5% to about 15% of an amphoteric surfactant selected from cocamidopropyl betaine, lauramidopropyl betaine, and combinations thereof, wherein the ratio of the anionic to the amphoteric surfactant is about 0.5:1 to about 1.5:1;
c. about 0.01% to about 2% of a cationic polymer having a charge density greater than 0.6 meq/g; and
d. at least 0.6% sodium chloride, wherein the composition is free of sulfated surfactants, has a ratio of polymer charge density to inorganic salt of about 1.2:1 to about 2:1, and lacks an in situ coacervate, as determined by the Microscopy Method to Determine Lack of In Situ Coacervate.
